(19) United States
(12) Patent Application Publication
(10) Pub. No.: US 2026/0272430 A1
(43) Pub. Date: Sep. 17, 2026

(54) FINE NEEDLE ASPIRATION (FNA) DEVICES AND METHODS INCLUDING STYLET EXTRACTION MECHANISMS

(71) Applicant: SEAN LYMAN, CHARLOTTESVILLE, VA (US)

(72) Inventor: SEAN LYMAN, CHARLOTTESVILLE, VA (US)

(21) Appl. No.: 19/566,367

(22) Filed: Mar. 13, 2026

Related U.S. Application Data
(60) Provisional application No. 63/771,465, filed on Mar. 13, 2025.

(51) Int. Cl. A61B 10/02; A61B 17/34; A61B 90/00

(52) U.S. Cl. CPC A61B 10/0283; A61B 17/3403; A61B 90/08; A61B 2010/0208; A61B 2017/3413; A61B 2090/0807

(57) ABSTRACT

A fine needle aspiration (FNA) system includes a reusable trigger gun having a support body and a trigger, and a removable extraction chamber/needle subassembly comprising an extraction chamber, an energy-storing element, a release mechanism configured to interface with the trigger, a needle hub, a needle, and a stylet. The extraction chamber/needle subassembly is detachably mounted to the reusable trigger gun, and actuation of the trigger releases the energy-storing element to retract the stylet into the extraction chamber. The extraction chamber/needle subassembly is detachable from at least the needle hub such that the subassembly can be detached while the needle remains inserted in a test subject.

### CROSS-REFERENCE TO RELATED APPLICATION

A claim of priority is made to U.S. Provisional Patent Application No. 63/771,465, filed Mar. 13, 2025, the disclosure of which is incorporated herein in its entirety.

### BACKGROUND

The inventive concepts generally relate to fine needle aspiration (FNA), and more particularly, the inventive concepts relate to FNA syringe and needle assemblies and to methods of using FNA syringe and needle assemblies.

Fine needle aspiration (FNA) is a common procedure used in the diagnosis of multiple medical problems, most commonly diagnosis of cancer. The procedure is performed using a fine gauge aspiration needle (usually 20-25 gauge) with a stylet. In particular, a hollow needle is typically configured with an engagement hub at one end, and a stylet may also be configured with an engagement hub at one end. The engagement hub of the needle is configured for coupling to and de-coupling from the engagement hub of the stylet, and also for coupling to and de-coupling from the hub of a syringe. In an engaged state, the stylet is inserted into the hollow needle with their engagement hubs coupled to one another. In some cases the stylet is devoid of a hub, in which case it may be held in place within the hollow needle by friction.

The FNA procedure may be carried out by inserting the hollow needle with the stylet into an abnormal soft tissue structure under ultrasound guidance, removing the stylet, and then attaching the syringe to the needle with the stylet removed. The stylet is intended to prevent or inhibit non-target cells from blocking the aspiration needle during insertion. Negative pressure is then applied to the syringe while moving the needle within the lesion, packing a lumen of the needle with cells which are then expelled onto slides for pathologic evaluation. Typically, the negative pressure is achieved by slowly withdrawing a plunger of the syringe.

One difficulty with the conventional method of obtaining fine needle aspirates is that the needle is held in one hand with the ultrasound probe in the other to insert the needle, but once the needle is in place the operator needs to put down the ultrasound probe in order to remove the stylet and attach the syringe. Then the probe must be picked up again and the needle and lesion relocated before the aspirate can be obtained.

Particularly in the case of very small lesions, this can add time and frustration to the procedure. In addition, the needle tip can be displaced from the lesion during removal of the stylet and attachment of the syringe leading to a suboptimal specimen.

Separately, in some cases syringe aspiration is not desired, either due to small size, increased vascularity, or tissue fragility. In these cases, the aspiration needle with the stylet in place is inserted under ultrasound guidance into the structure of interest, then the stylet removed and cells removed by capillary action and manual movement of the needle within the structure, packing the needle lumen with cells. Even in this case the ultrasound probe must be put down so the stylet can be removed without displacing the needle so a solution that would allow the operator to remove the stylet without losing ultrasound visualization of the needle tip would lead to increased biopsy yield and decreased procedure time.

### SUMMARY

According to an aspect of the inventive concepts, a syringe assembly is provided that includes a syringe body defining an internal cavity and a distal aperture configured to operatively receive a hollow needle and a stylet within the hollow needle, an extraction chamber disposed within the internal cavity, a plunger seal slidably engaging the syringe body to maintain an airtight seal, the plunger seal fixed to the extraction chamber, an actuation mechanism configured to retract the stylet from the hollow needle into the extraction chamber upon release of stored energy, and a trigger operatively coupled to the extraction chamber and the actuation mechanism, the trigger configured to activate the actuation mechanism to retract the stylet, and to translate the plunger seal relative to the syringe body so as to generate negative pressure within the internal cavity for aspiration.

According to another aspect of the inventive concepts, a trigger and extraction module configured for integration with a syringe body is provided. The module an extraction chamber dimensioned to receive a stylet in a retracted position, a plunger seal arranged to slide relative to the syringe body when installed, a spring coupled to a movable member, a trigger bar mounted for motion about a pivot and operatively coupled to and indirectly fixed to the plunger seal, and a locking element configured to restrain the spring in a preloaded state by engaging the movable member. Actuation of the trigger bar effects two sequential functions, namely: (i) first disengaging the locking element from the movable member to release the spring and retract the stylet into the extraction chamber, and (ii) then driving the plunger seal to aspirate a syringe cavity when the module is installed.

According to still another aspect of the inventive concepts, a method of performing fine needle aspiration using a syringe assembly is provided. The method include providing a syringe assembly comprising a syringe body, an internal extraction chamber, a plunger seal, a stylet extending through a needle, and a trigger operatively coupled to and indirectly fixed to the plunger seal, inserting the needle into target tissue with the stylet in a deployed position, actuating the trigger with the same hand holding the syringe body to perform first a retraction function that releases stored energy in an actuation mechanism and retracts the stylet into the extraction chamber; and then continuing actuation of the same trigger to move the plunger seal relative to the syringe body and thereby generate negative pressure within the syringe body to aspirate tissue through the needle.

According to another aspect of the inventive concepts, a device is provided which includes a syringe body, an extraction chamber configured to extract a stylet from a needle attached to the syringe body, an operator actuated member, wherein actuation of the operator actuated member causes the stylet to be extracted into the extraction chamber. Operator-actuated movement of the extraction chamber causes aspiration of a cavity of the body or of an operator targeted body structure.

According to yet another aspect of the inventive concepts, a syringeless fine needle aspiration device is provided which includes a needle and a needle hub, an extraction chamber, a stylet movable between a deployed position extending through the needle and a retracted position received within the extraction chamber, an energy-storing element coupled to the stylet, and an operator actuated trigger integrated with the extraction chamber and operatively coupled to a release mechanism. Actuation of the trigger releases the energy-storing element to retract the stylet into the extraction chamber; and the extraction chamber is releasably attachable to the needle hub such that the extraction chamber can be detached from the needle hub while the needle remains inserted in a test subject.

According to still another aspect of the inventive concepts, a system is provided which includes a reusable trigger gun having a support body and a trigger, and a removable extraction chamber/needle subassembly comprising an extraction chamber, an energy-storing element, a release mechanism configured to interface with the trigger, a needle hub, a needle, and a stylet. The extraction chamber/needle subassembly is detachably mounted to the reusable trigger gun, and actuation of the trigger releases the energy-storing element to retract the stylet into the extraction chamber. The extraction chamber/needle subassembly is detachable from at least the needle hub such that the subassembly can be detached while the needle remains inserted in a test subject.

According to another aspect of the inventive concepts, a method of performing fine needle aspiration without a syringe body is provided. The method includes mounting a removable extraction chamber/needle subassembly to a reusable trigger gun; inserting a needle of the subassembly into a test subject with a stylet in a deployed position; actuating a trigger of the trigger gun to release a stored energy element and retract the stylet into an extraction chamber of the subassembly; and detaching the extraction chamber/needle subassembly from at least a needle hub while leaving the needle inserted in the test subject to permit capillary sampling.

According to yet another aspect of the inventive concepts, a device is provided that includes a trigger gun and an extraction chamber detachably mounted on and operatively coupled to the trigger gun, the extraction chamber configured to retract a stylet from a needle when actuated by the trigger gun.

According to still another aspect of the inventive concepts, a system is configured to retract a stylet into an extraction chamber in response to a single user operated control and to permit detachment of the extraction chamber while a needle remains inserted into a test subject.

According to still another aspect of the inventive concepts, a method is provided that include retracting a stylet into an extraction chamber and subsequently decoupling the extraction chamber while maintaining a needle in situ.

### BRIEF DESCRIPTION OF THE DRAWINGS

The various aspects and features of the inventive concepts will become readily apparent from the detailed description that follows, with reference to the accompanying drawings, in which:

FIG. 1 is a perspective view of a syringe casing according to one or more embodiments of the inventive concepts;

FIG. 2 is a perspective view of a plunger according to one or more embodiments of the inventive concepts;

FIGS. 3A, 3B and 4 are perspective views of a hollow needle and stylet according to one or more embodiments of the inventive concepts;

FIGS. 5A and 5B are perspective views of a syringe assembly according to one or more embodiments of the inventive concepts;

FIGS. 6A, 6B, 6C, 7A and 7B are perspective views of a syringe casing including a stylet extraction mechanism according to one or more embodiments of the inventive concepts;

FIG. 8 is a perspective view of a plunger including a stylet extraction mechanism according to one or more embodiments of the inventive concepts;

FIG. 9 is a cross-sectional view of a plunger including a stylet extraction mechanism according to one or more embodiments of the inventive concepts;

FIGS. 10A, 10B, 10C and 10D are perspective views of a plunger including a stylet extraction mechanism according to one or more embodiments of the inventive concepts;

FIGS. 11A and 11B are side views for reference in describing a spring clamp for retaining a stylet according to one or more embodiments of the inventive concepts;

FIGS. 12A, 12B, 12C and 12D are perspective views for reference in describing a stylet extraction trigger one or more embodiments of the inventive concepts;

FIG. 13 is a flowchart for reference in describing a method of Fine Needle Aspiration (FNA) according to one or more embodiments of the inventive concepts;

FIG. 14A is a cross-sectional view of a syringe body in an example embodiment of the inventive concepts;

FIG. 14B is a cross-sectional view taken along line I-I of FIG. 14A in an example embodiment of the inventive concepts;

FIG. 14C is a cross-sectional view taken along line II-II of FIG. 14A in an example embodiment of the inventive concepts;

FIG. 14D is a bottom view of the syringe body of FIG. 14A in an example embodiment of the inventive concepts;

FIG. 15 is a cross-sectional view of the syringe body of FIG. 14 containing an extraction chamber and plunger seal in an example embodiment of the inventive concepts;

FIG. 16A is a cross-sectional view of the syringe assembly of FIG. 15 in which the stylet is in a deployed position in an example embodiment of the inventive concepts;

FIG. 16B is a cross-sectional view of the syringe assembly of FIG. 15 in which the stylet is in a retracted position by action of a tension spring in an example embodiment of the inventive concepts;

FIG. 16C is a cross-sectional view of the syringe assembly of FIG. 15 in which the extraction chamber and plunger seal are in a retracted position in an example embodiment of the inventive concepts;

FIG. 17 is a cross-sectional view for reference in describing an example of a triggering mechanism having a compression spring in an example embodiment of the inventive concepts;

FIG. 18A is an enlarged view of section A of FIG. 17 in which the trigger is in a locked position according to an example embodiment of the inventive concepts;

FIG. 18B is an enlarged view of section A of FIG. 17 in which the trigger is in an armed position according to an example embodiment of the inventive concepts;

FIG. 19 is a cross-sectional perspective view for reference in describing an embodiment devoid of a syringe body in which the trigger is integrated with the extraction chamber according to an example embodiment of the inventive concepts;

FIG. 20A is a perspective side view illustrating a reusable trigger gun according to an example embodiment of the inventive concepts;

FIG. 20B is a view taken along line III-III of FIG. 20A according to an example embodiment of the inventive concepts;

FIG. 20C is a view taken along line IV-IV of FIG. 20A according to an example embodiment of the inventive concepts;

FIG. 21A is a perspective side view illustrating a reusable trigger gun having an extraction chamber/needle assembly mounted thereon according to an example embodiment of the inventive concepts;

FIG. 21B is a view taken along line III-III of FIG. 21A according to an example embodiment of the inventive concepts;

FIG. 21C is a view taken along line IV-IV of FIG. 21A according to an example embodiment of the inventive concepts;

FIG. 22A is a perspective view of a gun/extraction chamber assembly coupled to a needle assembly inserted into a test subject at a target location according to an example embodiment of the inventive concepts; and

FIG. 22B is a perspective view of a gun/extraction chamber assembly decoupled from the needle assembly still inserted into a test subject at a target location according to an example embodiment of the inventive concepts.

### DETAILED DESCRIPTION

The inventive concepts will now be described in detailed by way of exemplary embodiments of the inventive concepts. Like elements and components are given like reference numbers throughout the drawings. Further, the drawings are not necessarily drawn to scale, and relative dimensions may be exaggerated for clarity of the description.

As will be described below, the inventive concepts present an integrated device which combines an echogenic FNA needle with a retractable stylet and an aspiration syringe capable of operation with one hand. The device would permit operators to access the lesion, retract the stylet, and obtain an aspirate under suction under continuous sonographic control. This would decrease procedure time, improve sample yield, and increase patient safety by keeping the needle visible to the operator at all times.

In an embodiment, the device is a syringe with an extraction chamber which contains a mechanism to retract the stylet. Retraction may be actuated, for example, by a button or trigger on the syringe. In some embodiments, a syringe plunger is designed to surround this extraction chamber and would be operated in a manner familiar to those who perform FNA. In other embodiments, the extraction chamber is contained within the plunger itself.

The device may ship assembled. The operator would insert the needle into the lesion, press the button to retract the stylet, and then aspirate as usual to obtain the sample. The entire device would then be passed to the pathology technician, who would then remove the needle and create smears for pathologic evaluation. Typically a small amount of preservative is then used to rinse the syringe to remove any sample that was aspirated into the syringe during sample collection.

As will be explained in detail below, the syringe assembly of some embodiments of the inventive concepts generally includes a syringe casing, a plunger, a hollow needle, a stylet, and a stylet extraction mechanism.

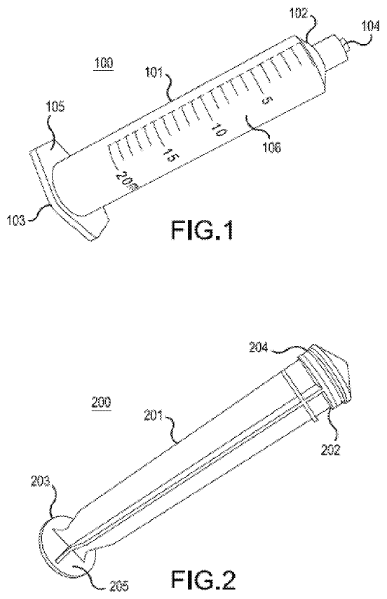

FIG. 1 is a perspective view of an example of a syringe casing 100. The syringe casing 100 of this example includes a barrel 101 defining an aspiration cavity 106 therein. The barrel 101 may be tubular (i.e., having a circular cross-section along its length) and include opposite first and second ends 102 and 103. In addition, the barrel 101 may include calibration markings (such as those shown in FIG. 1), and may be made of a transparent or semi-transparent material. At the first end 102 of the barrel 101, the syringe casing 100 may include an adaptor or hub 104 for connection to a needle assembly (described below). At the second end 103 of the barrel 101, the syringe casing 100 may include one or more flanges 105 to assist in manual manipulation of the syringe assembly during use. The syringe casing 100 is presented here as an example only, and embodiments of the inventive concepts are not limited to the particularities of the syringe casing 100.

FIG. 2 is a perspective view of an example of a plunger 200. The plunger 200 of this example includes a plunger body 201 having opposite first and second ends 202 and 203. The plunger body 201 is configured for insertion into the syringe casing 100, and is movable within the barrel 101 of the syringe casing 100 to define a volume of the aspiration cavity 106. At the first end 202 of the plunger body 201 is a plunger seal 204. The plunger seal 204 is configured to provide an air-tight seal at the inner surface of the barrel 101. In this manner, positive air pressure may be exerted in the aspiration cavity 106 by moving the plunger towards the first end 102 of the barrel 101, and negative pressure may be exerted in the aspiration cavity 106 by extracting the plunger 200 towards the second end 103 of the barrel 101. In the example of FIG. 2, the plunger 200 includes a flange 205 to assist in manual manipulation of the plunger 200 relative to the syringe casing 100. Also in the example of FIG. 2, the plunger body 201 has a +-shaped cross-section along its length. However, the plunger 200 of FIG. 2 is presented here as an example only, and embodiments of the inventive concepts are not limited to the particularities of the plunger 200.

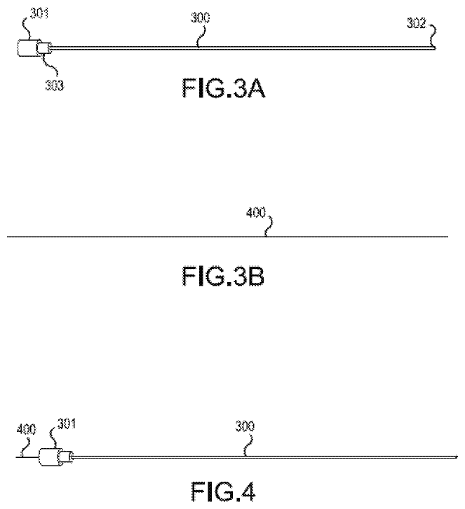

FIGS. 3A, 3B and 4 are perspective views for reference in describing examples of a hollow needle 300 and stylet 400. In particular, FIG. 3A is a perspective view of a hollow needle 300, FIG. 3B is a perspective view of a stylet 400, and FIG. 4 is a perspective view of the hollow needle 300 and stylet 400 in their assembled state. Referring collectively to these figures, a distal end 302 of the needle 300 may be configured for piercing tissue, and a proximal end 303 of the hollow needle 300 may be coupled to a hub 301. The hub 301 is configured for attachment to the hub 104 of the syringe casing 100. When attached, a lumen of the hollow needle 300 is in fluid communication with the aspiration cavity 106 of the syringe casing 100. The stylet 400 is configured for insertion through the hollow needle 300. The stylet 400 is intended to prevent or inhibit non-target cells from blocking the hollow needle 300 during insertion into tissue. In the assembled state (FIG. 4), the stylet 400 has a distal portion extending to the distal end 302 of the hollow needle 302 and a proximal portion extending within the syringe casing 100 (FIG. 1). As will be explained later, an extraction mechanism is located within the syringe casing 100 and is coupled to the proximal end of the stylet 400.

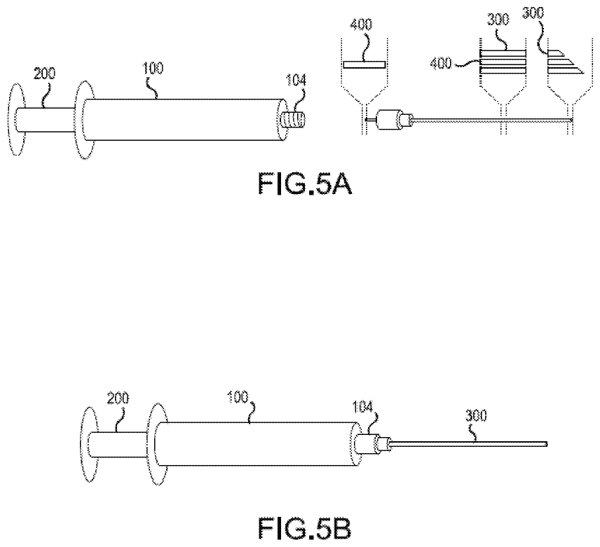

FIGS. 5A and 5B are perspective views for reference in describing an assembled syringe assembly according to example embodiments of the inventive concepts. In particular, FIG. 5A is a perspective view of the hollow needle 300/stylet 400 separated from the syringe casing 100. Although FIG. 5A illustrates the distal end of the stylet 400 aligned at a slant with the distal end of the hollow needle 300, the embodiments of the inventive concepts are not limited in this manner.

FIG. 5B is a perspective view of the syringe assembly with the needle 300/stylet 400 attached to the syringe casing 100. In the embodiment of FIG. 5B, the stylet 400 is not externally exposed. On other embodiments, however, a distal end of the stylet 400 may protrude slightly past a distal end of the hollow needle 300.

Although not visible in FIG. 5B, the proximal end of the stylet 400 is attached to an extraction mechanism located within the syringe assembly. In some embodiments of the inventive concepts, the extraction mechanism is spring-loaded and activated by a trigger accessible to the operator. Also, in some embodiments of the inventive concepts, the extraction mechanism is located in the plunger 200. In other embodiments of the inventive concepts, the extraction mechanism is located in the syringe casing 100.

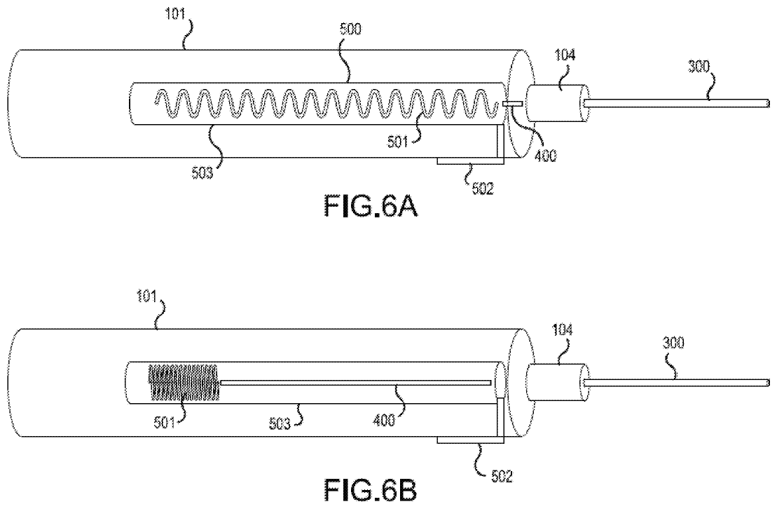

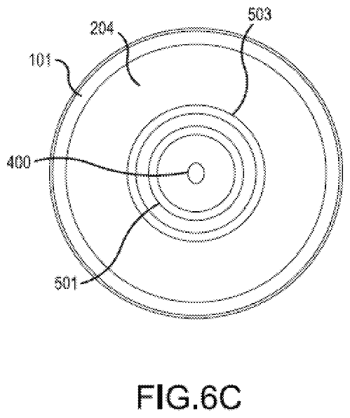

FIGS. 6A and 6B are perspective views for reference in describing an embodiment in which the extraction mechanism 500 is located in the syringe casing 100. In this embodiment, extraction mechanism 500 includes an extraction chamber 503 that is fixed to one or more inside walls of the syringe casing 101. The extraction chamber 503 may be tubular to conform to the shape of an extension spring 501 contained therein, and may extend coaxially within the barrel 101 of the syringe casing 100. The manner in which the extraction chamber 503 is fixed to the inner wall(s) of the syringe casing 100 can be varied by design, so long as fluid communication is not blocked between the hollow needle outlet of the hub 104 and the aspiration cavity 106 of the syringe casing 100.

FIG. 6A represents a state in which the stylet 400 extends through the hollow needle 300 (i.e., a state in which the hollow needle 300 is inserted into tissue). As shown, a proximal end portion of the stylet 400 is connected to one end portion of an extension spring 501 contained within the extraction chamber 503. The other end portion of the extension spring 501 is fixed in place at an opposite end of the extraction chamber 503. In this embodiment, the extension spring 501 is stretched along its length to generate a tensile load and held in place by a lever or latch or other mechanism. FIG. 6B represents a state in which the tensile load of the extension spring 501 is released by depressing a trigger (or button) 502 which detaches the extension spring 501 from the lever or latch or other mechanism that was holding the spring in place as in FIG. 6A. As a result, the extension spring 501 retreats to its non-loaded state, drawing the stylet 400 into the extraction chamber 503.

The plunger 200, which is not shown in FIGS. 6A and 6B, may include an aperture or opening along its length to allow it to travel along the length of the extraction chamber 503. In this case, referring to the cross-sectional view of FIG. 6C, the plunger seal 204 may have an outer circumferential sealing surface in contact with an inner wall of the barrel 101 of the syringe casing 100, and an inner circumferential sealing surface in contact with an outer wall of extraction chamber 503. Also represented in FIG. 6C is the spring 501 and stylet 400 contained with the extraction chamber 503.

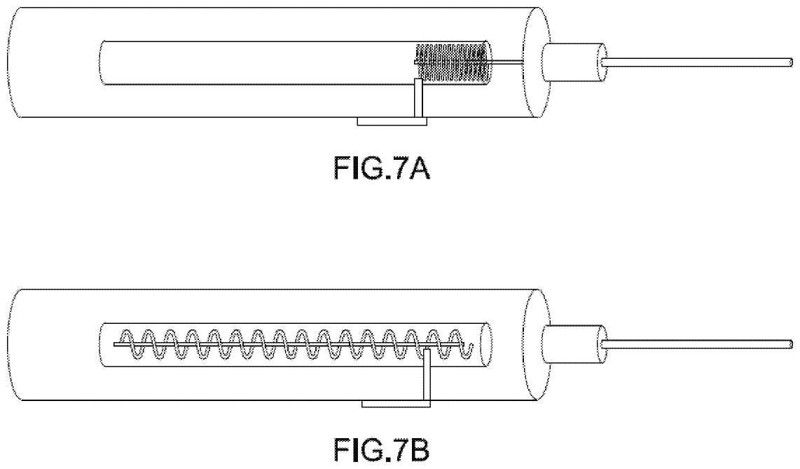

FIGS. 7A and 7B are perspective views for reference in describing another embodiment in which the extraction mechanism 500 is located in the syringe casing 101. This embodiment is the same as that of FIGS. 6A and 6B, except that the spring 501 is a compression spring instead of an extension spring. As such, as shown in FIG. 7A, the spring 501 is compressed in its initial state with the stylet 400 extending through the needle 300. A resultant compressive load is released by depressing the trigger 502, causing the spring 501 to expand to its non-loaded state, thus drawing the stylet 400 into the extraction chamber 503.

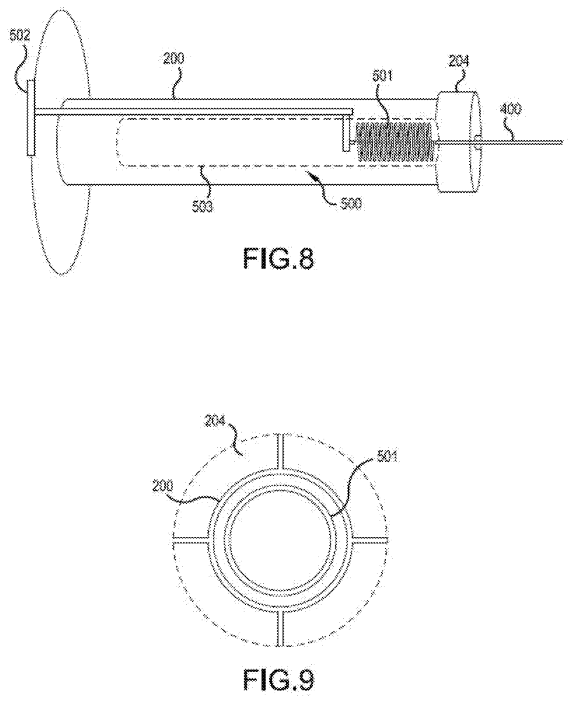

As mentioned above, in some embodiments of the inventive concepts, the extraction mechanism 500 is located in the plunger 200. An example of this is represented in FIG. 8 of the drawings. As shown, the extraction mechanism 500 may be embedded within the plunger 200. FIG. 8 depicts an example in which the extraction mechanism 500 includes an extraction chamber 503 and a compression spring 501 like that described above in connection with FIGS. 7A and 7B. In an alternative embodiment, the spring 501 may be an expansion spring like that of FIGS. 6A and 6B. In either case, a trigger 502 is located on the plunger to release a load (compression or expansion) of the spring 501 and draw the stylet 400 into the casing 503. Although not shown in FIG. 8, the plunger 200 is inserted in the syringe casing 101 with the stylet 400 extending through the hub 104 into the hollow needle 300.

In some embodiments, a separate chamber 503 is omitted. For example, referring to the cross-sectional view of FIG. 9, the inner walls of the plunger 200 may house the spring 501. In other words, a chamber of the plunger 200 may function as the extraction chamber. FIG. 9 also depicts a representation of the plunger seal 204 and ribs which may extend lengthwise along the outer diameter of the plunger 200. However, the inventive concepts are not limited to such a configuration.

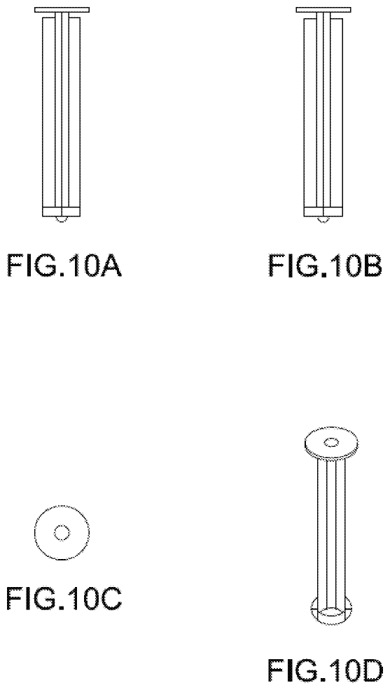

FIGS. 10A, 10B, 10C and 10D are additional perspective views of the plunger 200 including a stylet extraction mechanism. In particular, FIGS. 10A and 10B are side-views, FIG. 10C is a bottom view, and FIG. 10D is a perspective view. In this illustrated example, a flange is located at the top of the plunger 200, a needle/stylet attachment surface is located at the bottom of the plunger 200, and a stylet extraction chamber is located there between.

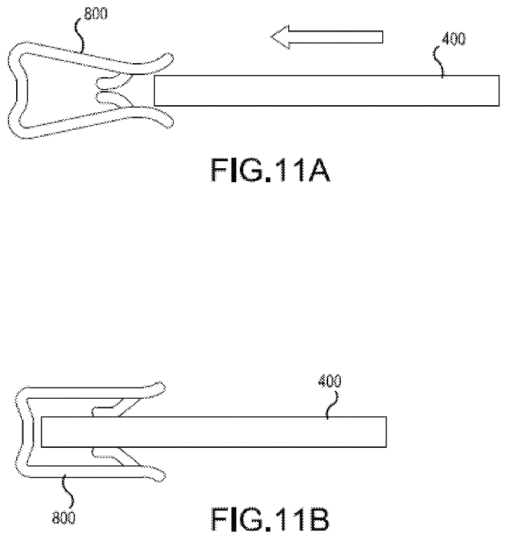

In some embodiments of the inventive concepts, the syringe assembly is prepackaged in a fully assembled state prior to use. In other embodiments of the inventive concepts, the needle 300/stylet 400 are attached to the syringe assembly by the operator (medical professional). Referring to FIG. 5A, this may be done by the operator inserting the stylet 400 into the proximal end of the syringe casing 100. The extraction mechanism of the syringe may be configured with a locking mechanism (operatively coupled to the extraction spring) configured to receive and lock-in-place the proximal end of the stylet 400. One example of such a locking mechanism is a ribbed channel configured to allow to stylet 400 to be inserted therein, but with sharp angular ridges or teeth configured to inhibit the stylet 400 from being detached after insertion. Another example of a locking mechanism is illustrated in FIGS. 11A and 11B. Here, a spring clamp 800 is configured to expand as the proximal end of the stylet 400 is pushed into the clamp 800. The style 400 remains seated in the clamp 800 primarily due to the clamping spring action of the clamp 800. If desired, one or more surfaces of the spring clamp 800 in contact with the stylet 400 can be roughened or ribbed to increase the frictional forces acting on the stylet 400. Further, although not shown, guide members can be included to direct the proximal end of stylet 400 into the spring clamp 800 (or other locking mechanism) when inserted into place. The inventive concepts are not limited by the particularities of the locking mechanism.

The embodiments described above utilize the stored energy of a compression or expansion spring or coil to extract the stylet from hollow needle. The inventive concepts, however, are not limited in this manner. For example, other types of springs may be utilized to draw the stylet from the hollow needle, such as torsion spring. Further, in addition to or in place of mechanical extraction mechanisms, electrical and/or magnetic mechanisms may be utilized to extract the stylet. In other words, any operator-triggered mechanism capable of storing the energy necessary to extract the stylet from the hollow needle may be utilized.

The location of the trigger (or button) used to activate the extraction mechanism is a matter of design. Ergonomically, it may be advantageous to place the trigger at a particular location, such as close to the needle-end of the syringe casing or at the push surface of the plunger. However, the inventive concepts are not limited to the location of the trigger.

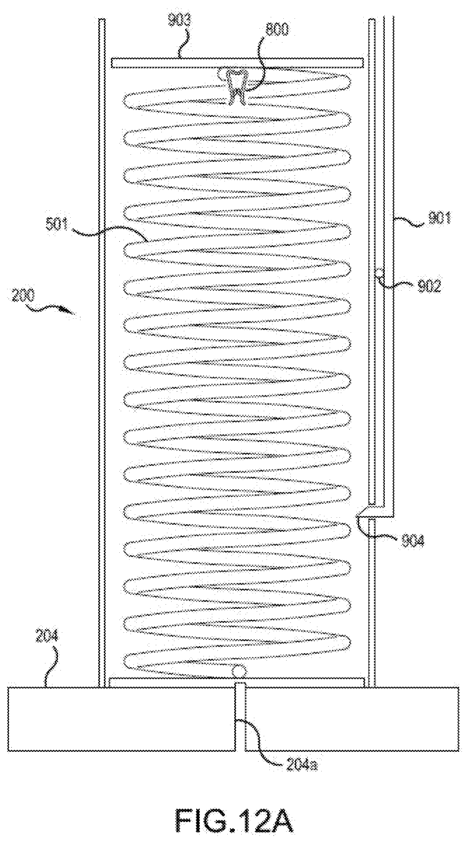

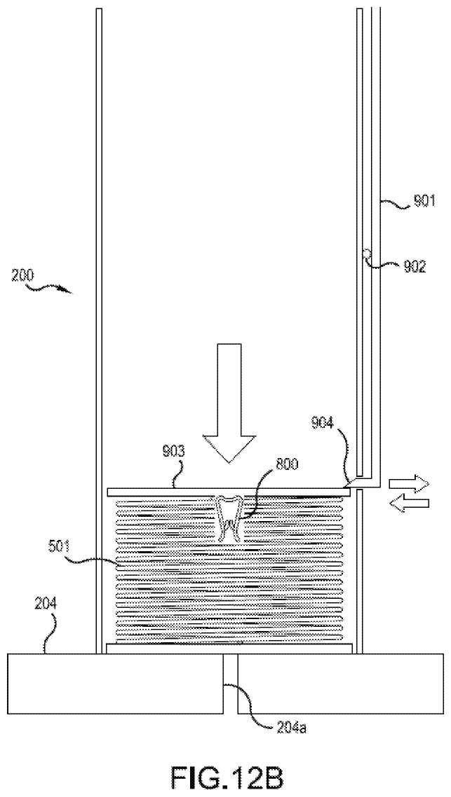

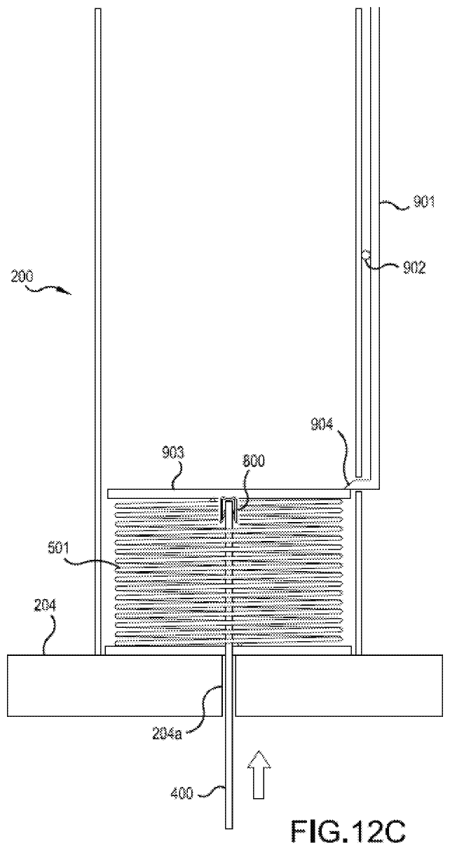

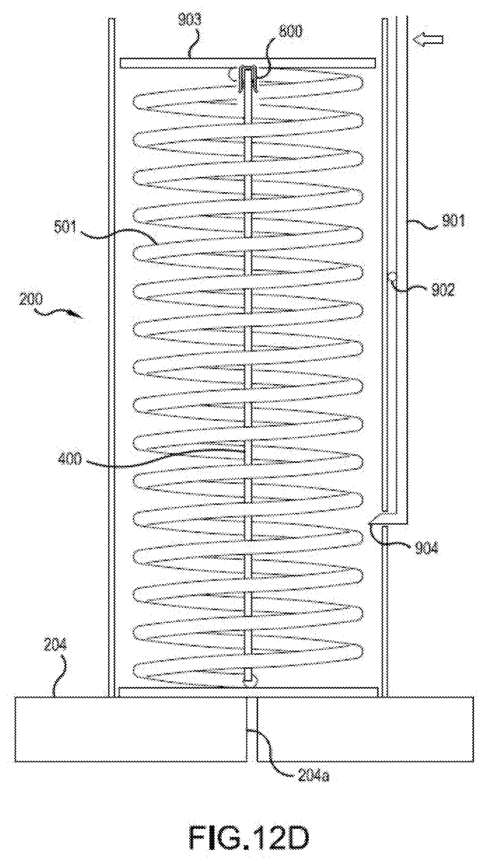

The inventive concepts are also not limited by the configuration of the trigger, and as those skilled in the art will appreciate, there are any number of mechanisms that can be conceived to release the potential energy of the spring fixed to the stylet. Nonetheless, FIGS. 12A, 12B, 12C and 12D illustrate a simple example in which a trigger bar 901 extends along the length of the plunger 200 and is pivotably mounted at a pivoting element 902 located on an outer sidewall of the plunger 200. FIG. 12A illustrates a “pre-loaded” state in which the expansion spring 501 is in its expanded and relaxed state, and the stylet is not yet engaged. As shown in FIG. 12B, the expansion spring 501 is set to a locked position by pressing an upper surface of a bar or plate 903 having a lower surface fixed to the spring 501. The bar or plate 903 forces an angled surface of the locking element 904 (and the trigger bar 901) outwardly as it is pushes pass the locking element 904, after which the locking element 904 returns to is initial position to lock the spring 501 in place as represented in FIG. 12B. The return of the locking element 904 to its initial position can be carried out manually by the operator, or automatically resulting from a spring action of the trigger bar 901 and/or pivot element. It is also noted that the expansion spring 501 can be set to its locked position either at the assembly factory prior to shipment, or by the operator prior to the FNA procedure. Next, as represented in FIG. 12C, the stylet 400 may be inserted through a central aperture 204a in the plunger seal 204 and then into a spring clamp 800 fixed to a lower surface of the bar or plate 903 attached at the one end of the spring 501. In this state, the extraction mechanism of the syringe assembly is ready for the FNA procedure. FIG. 12D represents a state in which the trigger mechanism has been activated by pressing the other end of the trigger bar 901. Pushing the other end of the trigger bar 901 causes the locking element 904 to withdraw from the plunger 200, thus releasing (or unlocking) the spring 501. As a result, the spring 501 expands as represented in FIG. 12D to extract the stylet 400 into the plunger 200. In this state, the plunger 200 may have a cross-section such as that shown in previously described FIG. 9.

As explained previously, in conventional arrangements, the hollow needle and the syringe are initially separated from one another at the start of the FNA procedure, and only later is the syringe attached to hollow needle during the FNA procedure (i.e., after extraction of the stylet). On the other hand, in some example embodiments of the inventive concepts, the hollow needle is attached in advance to the syringe. For example, the syringe assembly with the attached hollow needle may be pre-packaged as a single ready-for-use unit. That is, the syringe may be fully assembled and packaged inside a box or other enclosure. In this case, a removable sheath may cover the otherwise exposed hollow needle as a safety precaution. In practice, the operator would remove the syringe assembly from the package, remove the sheath (if any) covering the needle, and proceed with the FNA procedure. Other than removal of the sheath, the syringe assembly is ready for use when removed from the package. In this case, the extraction mechanism may be locked in a loaded position with the stylet extending through the hollow needle. Alternatively, the operator may lock the extraction mechanism in its loaded position after removing the syringe assembly from the package. Also, in some embodiments of the inventive concepts, the syringe assembly is a one-time use device that is disposed of after the FNA procedure.

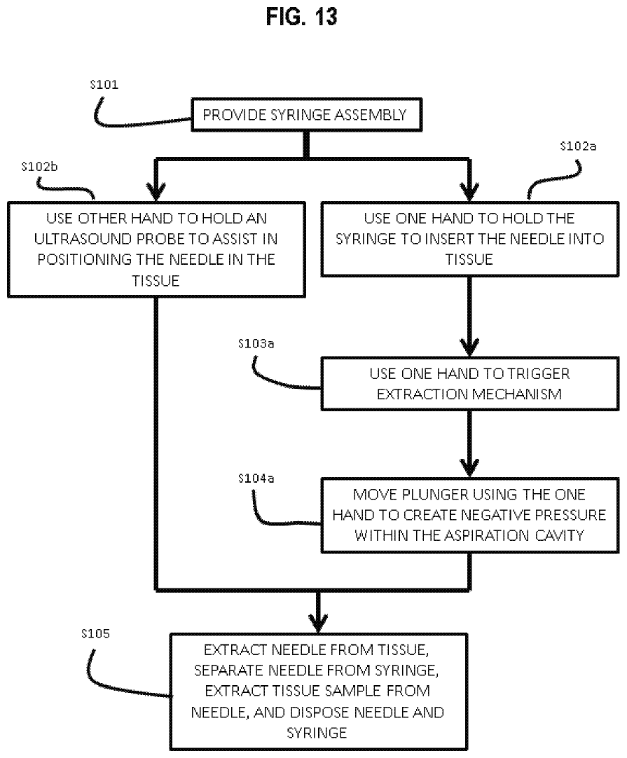

The inventive concepts include a FNA method such as that represented by the flowchart of FIG. 13. Referring to FIG. 13, the FNA method includes providing a syringe assembly (S101) according to one or more of the embodiments described above. The method further includes using one hand to hold the syringe to insert the needle into tissue (S102a) while using another hand to hold an ultrasound probe to assist in positioning the needle in the tissue (S102b). The method further includes triggering the button using the one hand (S103a) to cause the extraction mechanism to fully extract the stylet from the needle into the extraction chamber of the syringe while continuing to hold the ultrasound probe in the other hand, and moving the plunger using the one hand (S104a) to create negative pressure within the aspiration cavity while continuing to hold the ultrasound probe in the other hand, wherein the negative pressure causes tissue sample to be drawn into the needle. The method further includes extracting the needle from the tissue, separating the needle from the syringe, extracting the tissue sample from the needle and/or syringe, and optionally disposing of the needle and syringe (S105). That is, in some embodiments of the inventive concept, the syringe assembly is a single-use device that is disposed of after the FNA specimen is obtained.

As noted previously, the drawings are not necessarily drawing to scale. On a related note, the embodiments described herein are applicable to various needle gauges and lengths, as well as various syringe lengths and volumes. Different sizes/lengths of the syringe assembly parts and volumes may be preferred depending on tissue types and lesion locations. Further, in alternative embodiments, the extraction chamber and the stylet may be longer than the barrel of the syringe and/or longer than the plunger (i.e., the extraction chamber may extend past the tops of the syringe and/or plunger). Still further, in other alternative embodiments, the hollow needle is longer than the barrel of the syringe, but the extraction chamber (and stylet when extracted) is fully contained within the barrel of the syringe or within the plunger. In this case, for example, a monofilament may be attached on one end to a stylet within the hollow needle and on the other to a small cylinder in the extraction chamber. That cylinder may be locked in place and attached to a loaded spring that when unlocked results in rotation of the cylinder, wrapping the monofilament and retracting the stylet. This allows for the use of a stylet that is shorter than the hollow needle.

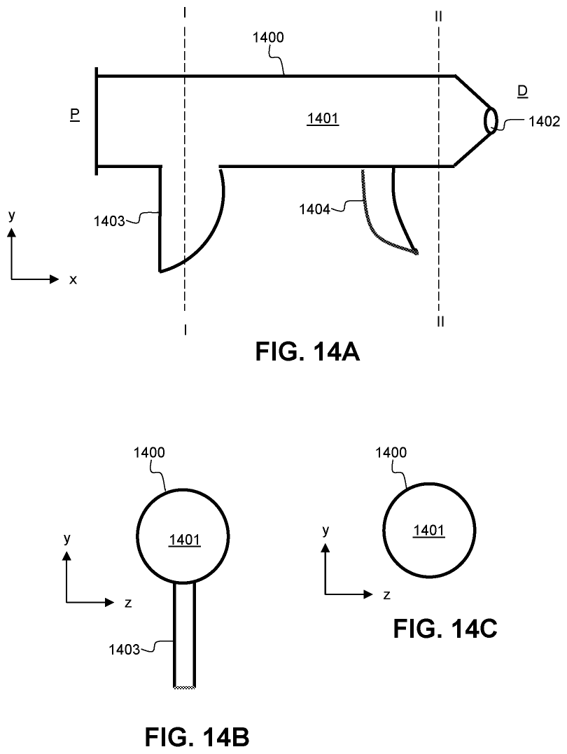

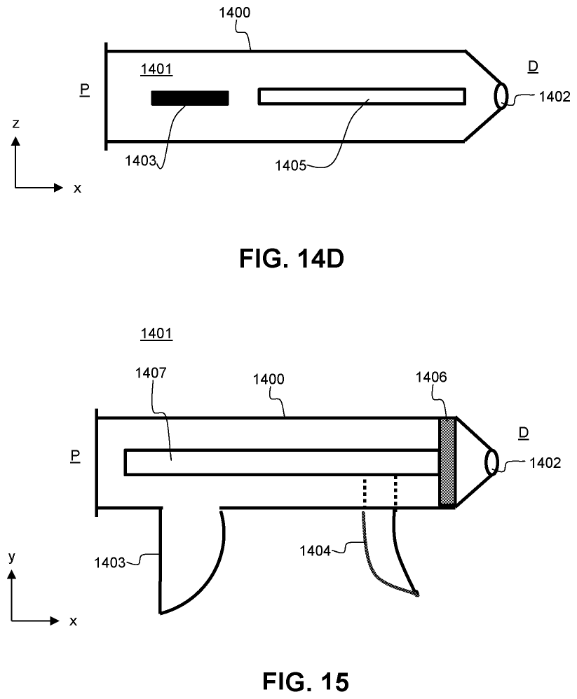

One or more other embodiments of the inventive concepts will now be described with reference to FIGS. 14A through 18B.

FIG. 14A illustrates a syringe body 1400 defining an internal syringe cavity 1401 extending between the proximal end P and distal end D. The distal end includes a needle assembly aperture 1402 sized to receive a conventional FNA needle hub or related coupling mechanism. A handle 1403 extends laterally from the syringe body and provides a grip surface configured for one handed operation. A trigger 1404 is positioned on or adjacent to the handle 1403, enabling the operator to actuate an internal stylet extraction mechanism while maintaining continuous ultrasound guidance.

FIG. 14B shows the internal geometry of the syringe body 1400 and cavity 1401 when sectioned along line II of FIG. 14A. This view highlights the lateral orientation of handle 1403 and the mounting structure of trigger 1404 relative to the cylindrical body. It also shows that the cavity 1401 extends axially and remains unobstructed for reception of a plunger assembly and extraction chamber.

FIG. 14C provides a cross-sectional view orthogonal to FIG. 14B, showing the syringe body geometry along line IIII. This view highlights the alignment between the trigger 1404, trigger channel 1405, and the cavity 1401, confirming that the trigger force is transmitted along a defined internal path for actuating downstream components.

FIG. 14D shows the bottom of syringe body 1400, including the bottom surfaces of cavity 1401, aperture 1402, and handle 1403. The trigger channel 1405 is visible from beneath, showing a recessed linear passage that guides movement of the trigger 1404 or its associated trigger bar. This bottom view also illustrates the distal orientation of the aperture 1402 relative to handle 1403, optimizing ergonomic alignment with the trigger.

FIG. 15 shows the syringe assembly of FIG. 14 with an extraction chamber 1407 and a plunger seal 1406 positioned within cavity 1401. The extraction chamber 1407 extends axially from the proximal region toward the distal region and is dimensioned to receive a retractable stylet. The plunger seal 1406 surrounds the proximal portion of the extraction chamber 1407, providing an airtight sliding interface with both the syringe cavity 1401 and extraction chamber 1407.

The trigger 1404, when actuated, ultimately interacts with a spring biased extraction mechanism housed within or coupled to the extraction chamber 1407.

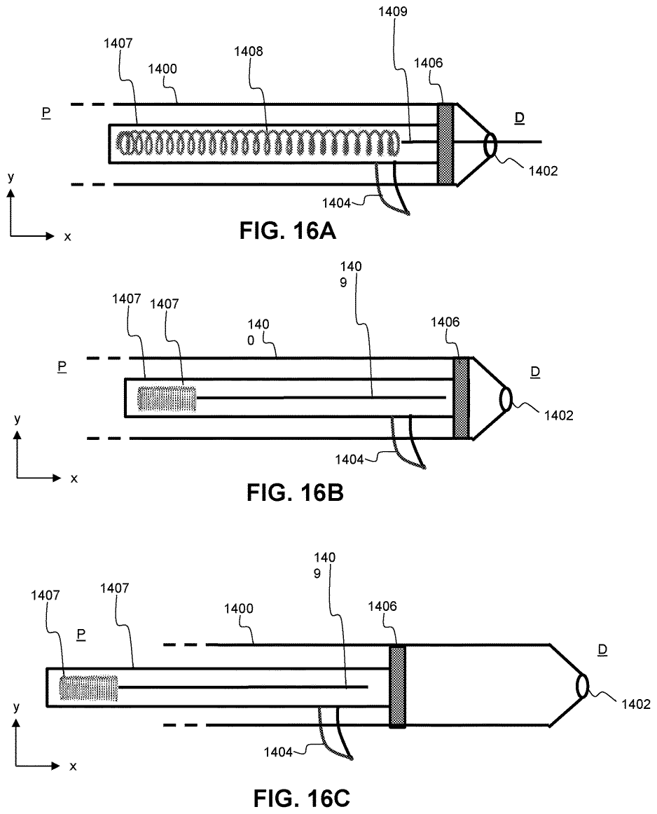

FIG. 16A shows the stylet 1409 in its deployed (distal) position extending through aperture 1402. The tension spring 1408 is shown in a loaded state within extraction chamber 1407. The plunger seal 1406 remains at its initial proximal position, and trigger 1404 has not yet been actuated.

FIG. 16B illustrates activation of the extraction mechanism. Upon actuation of trigger 1404, the tension spring 1408 contracts, drawing the stylet 1409 proximally into extraction chamber 1407. This results in complete retraction of the stylet from the hollow needle, enabling immediate aspiration without requiring two-handed manipulation.

In FIG. 16C, the extraction chamber 1407 and plunger seal 1406 have moved proximally relative to syringe body 1400. This may correspond to operator-initiated plunger motion to generate negative pressure, or a result of spring mediated recentering following the stylet retraction event. Ideally, however, the plunger would not move when the retraction device is activated. That is, in an optimal process the operator alone would control plunger retraction, and there would be little to no movement of the plunger during stylet extraction. Regardless, once extracted, the stylet remains housed entirely within extraction chamber 1407.

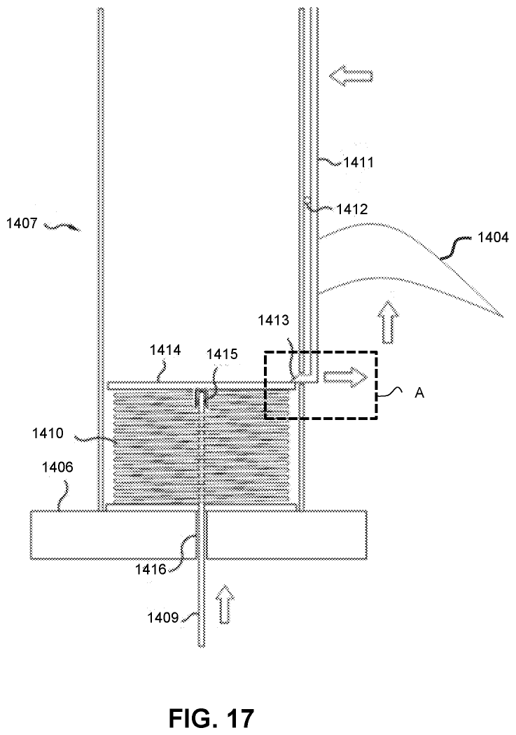

FIG. 17 illustrates a detailed embodiment of the trigger and locking mechanism used to actuate extraction. A compression spring 1410 is positioned between extraction chamber 1407 and a plate 1414 coupled to the trigger bar 1411. The trigger bar 1411 is pivotably mounted via pivoting element 1412, enabling rotational motion of the bar when the trigger 1404 is pressed. A locking element 1413 engages the plate 1414 to maintain the compression spring 1410 in a loaded position until trigger actuation. A spring clamp 1415 retains the proximal end of the stylet 1409 within the extraction mechanism, and a plunger seal aperture 1416 allows passage of the stylet during loading and retraction.

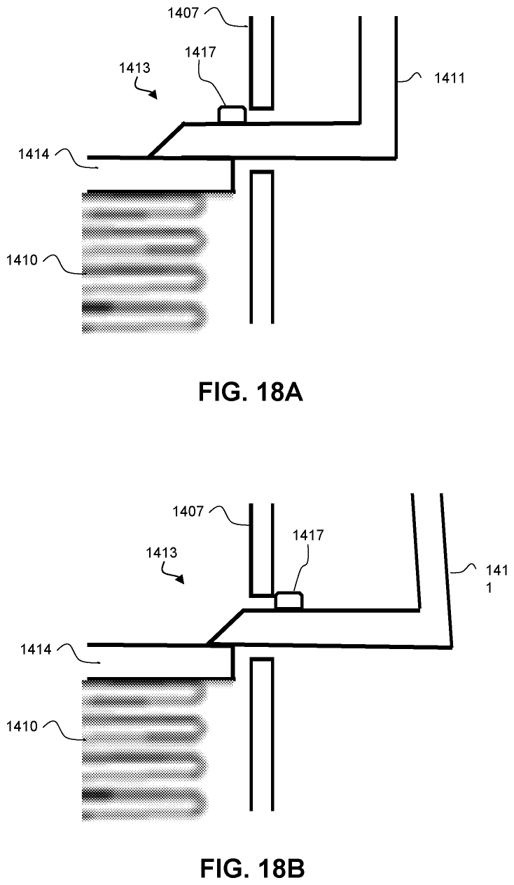

FIG. 18A depicts the mechanism in a locked state. The locking element 1413 engages with plate 1414, preventing movement of the compression spring 1410. The trigger bar 1411 is held away from initiating release, and stop nodule 1417 maintains alignment of locking components. This locked configuration maintains the spring 1410 in a preloaded condition prior to use.

FIG. 18B shows the armed configuration. When the trigger 1404 is pressed, the trigger bar 1411 rotates about pivoting element 1412, causing locking element 1413 to disengage from plate 1414. This releases compression spring 1410, allowing it to drive proximal motion of plate 1414, which in turn retracts the stylet 1409 into extraction chamber 1407. The stop nodule 1417 helps guide the transition from locked to armed state and limits overtravel.

This locking mechanism enables a secure, single hand-activated release that prevents accidental stylet retraction while the device is being positioned.

It should be understood that the particular trigger mechanism illustrated in FIGS. 17, 18A, and 18B—including the configuration of the trigger bar 1411, pivoting element 1412, locking element 1413, plate 1414, compression spring 1410, and stop nodule 1417—is presented merely as one nonlimiting example of a suitable actuation system for releasing stored potential energy within the extraction mechanism.

Those of ordinary skill in the art will readily appreciate that numerous alternative trigger and release mechanisms may be employed to achieve the same functional result of controllably releasing a stored mechanical load (e.g., compression, tension, torsion, or magnetic energy) to retract the stylet into the extraction chamber. For example, without limitation, suitable alternatives may include:
sliding or pushbutton triggers,
cam or detent-based release mechanisms,
rotary levers,
overcenter spring releases,
latch and pawl systems,
magnetically or electrically actuated release members,
or any other structure capable of selectively releasing stored energy in response to operator input.

Accordingly, the trigger assemblies disclosed herein should be understood as illustrative rather than limiting, and any operator actuated mechanism capable of releasing the extraction spring or equivalent energy storing element falls within the scope of the inventive concepts.

In some embodiments, the device is provided as a syringe assembly including a syringe body 1400 that defines an internal syringe cavity 1401, a distal needle assembly aperture 1402, a handle 1403, and a trigger 1404 arranged to move within a trigger channel 1405..pdf) The extraction chamber 1407 and a plunger seal 1406 are mounted within the syringe cavity 1401 such that the plunger seal slidingly seals both against the inner wall of the syringe body and around the extraction chamber.

Prior to patient contact, a hollow needle with a stylet 1409 is coupled to the distal aperture 1402. The stylet 1409 extends distally in a deployed position and is operatively coupled at its proximal end to the extraction mechanism housed within or on the extraction chamber 1407 (for example, by a spring clamp 1415 attached to a plate 1414). The extraction mechanism is preloaded by a spring, which may be a tension spring 1408 or a compression spring 1410, and is secured by a locking element 1413 that is releasable via movement of a trigger bar 1411 about a pivoting element 1412.

Under ultrasound guidance, the clinician localizes the target lesion while holding the ultrasound probe in one hand and the syringe assembly by the handle 1403 in the other. The ergonomic relationship of the handle, trigger 1404, and trigger channel 1405 enables singlehanded operation of the retraction and aspiration sequence while maintaining sonographic visualization.

With the stylet 1409 in the deployed position, the clinician advances the needle through the patient's tissue toward the lesion, with the stylet tip protecting the needle lumen from occlusion by non-target tissue. The assembly at this stage corresponds to the configuration depicted in FIG. 16A, where the stylet extends through aperture 1402 and the extraction mechanism remains preloaded.

When the needle tip is situated within the lesion, the clinician actuates the trigger 1404. The trigger is operatively coupled to, and indirectly fixed to, the plunger seal 1406, and its motion is guided along the trigger channel 1405. Initial travel of the trigger bar 1411 about the pivot 1412 disengages the locking element 1413 from the plate 1414, thereby releasing stored energy of the spring (e.g., compression spring 1410 or tension spring 1408) to retract the stylet 1409 proximally into the extraction chamber 1407, as illustrated in FIGS. 16B and 17-18B..pdf)

In some embodiments, a lost motion or first segment travel is provided such that the retraction event occurs without appreciable displacement of the plunger seal 1406, ensuring that the stylet retracts and the needle lumen opens before aspiration commences. The stop nodule 1417 and associated guide surfaces can define a locked and an armed state of the locking element preceding release.

Upon continued movement of the same trigger 1404 along the trigger channel 1405, the linkage to the plunger seal 1406 engages a second segment travel that translates the plunger seal within the syringe cavity 1401. This translation increases the volume of the cavity and generates negative pressure to aspirate tissue or cellular material through the now open needle lumen into the syringe system, as shown schematically by the relative movement states in FIG. 16C..pdf)

With the stylet retracted and aspiration underway, the clinician may modulate aspiration by controlling the extent and rate of trigger movement that drives the plunger seal 1406, thereby adjusting negative pressure within cavity 1401 while maintaining continuous ultrasound visualization of the needle tip. The handle 1403 position and trigger channel 1405 geometry allow stable, linear or guided arcuate input to produce predictable plunger seal displacement.

If desired, the clinician may perform short, controlled needle motions within the lesion to augment capillary draw while aspiration is applied, packing the needle lumen with target cells. Because the trigger provides a singlehanded sequential function—first stylet retraction, then aspiration—the opposite hand can remain on the ultrasound probe throughout.

After sufficient material is obtained, the clinician ceases trigger motion to stabilize the plunger seal 1406, withdraws the needle from the tissue, and detaches the needle assembly at aperture 1402. The specimen can then be expressed from the needle and/or syringe for cytologic preparation according to standard practice. The extraction chamber 1407 retains the retracted stylet 1409 during these steps, reducing the risk of accidental redeployment.

While the foregoing describes a sequence using a compression spring 1410 based trigger release (e.g., FIG. 17 and FIGS. 18A-18B), the same procedural steps apply when a tension spring 1408 provides the retraction force (e.g., FIGS. 16A-16B), and when the trigger linkage or geometry differs, provided that a first trigger motion releases the retraction mechanism and a continued trigger motion translates the plunger seal to aspirate. The trigger channel 1405 can be linear or arcuate; the locking element 1413 may be a latch, pawl and detent, overcenter linkage, cam-based detent, or electrically or magnetically actuated catch, in each case configured to be disengaged by initial trigger motion and followed by plunger seal translation.

In some embodiments, the device may include adjustable stops or biasing features that set the amount of lost motion during the first segment travel to tailor retraction timing relative to aspiration onset, and/or limit total plunger seal stroke to a desired aspiration volume for a given procedure.

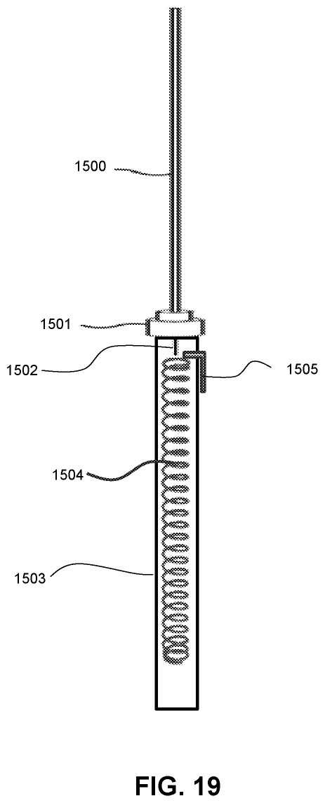

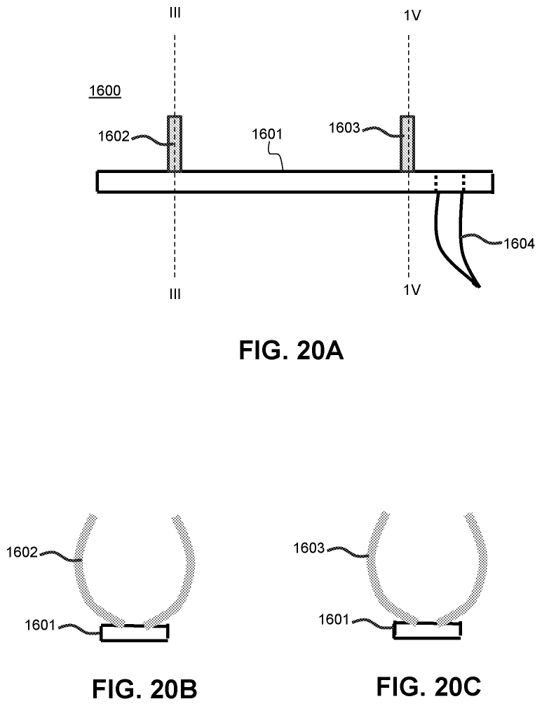

Attention will now be focused on the embodiments of FIGS. 19-22B. These embodiments omit a syringe body and instead combine a stylet retraction mechanism with a standalone extraction chamber that can either include its own trigger (FIG. 19) or mount onto a reusable trigger gun (FIGS. 20A-22B). In use, an operator inserts an FNA needle with the stylet in place, retracts the stylet via a trigger, and can then decouple the extraction chamber while leaving the needle in situ for capillary based FNA sampling under continuous ultrasound visualization.

FIG. 19 depicts a compact assembly comprising a needle 1500 coupled to a needle hub 1501, a stylet 1502, an extraction chamber 1503, a tension spring 1504, and a trigger 1505. The trigger 1505 releases stored energy in spring 1504 to retract the stylet 1502 proximally into extraction chamber 1503, thereby opening the needle lumen for sampling. In the approach state, stylet 1502 extends through needle 1500 to prevent nontarget material from occluding the lumen during insertion.

Importantly, in this embodiment the needle/needle hub (1500/1501) is detachable from extraction chamber 1503 even while the needle remains inserted in the test subject, allowing the operator to set aside the retracted stylet/chamber component without disturbing needle position and to perform capillary FNA within the lesion.

While FIG. 19 illustrates a tension spring 1504, other energy storage and release mechanisms (e.g., compression or torsion springs, or magnetically/electrically latched releases) may be employed, provided that trigger 1505 controllably retracts the stylet 1502 into chamber 1503.

FIG. 20A shows a reusable trigger gun 1600 with support body 1601, proximal attachment clip 1602, distal attachment clip 1603, and trigger 1604. The gun 1600 accepts and operates compatible extraction chamber/needle subassemblies for singlehanded use.

FIG. 20B and FIG. 20C illustrate how clips 1602/1603 mechanically capture the extraction chamber housing and align its release interface to the gun's trigger 1604. The clips support rapid loading/unloading and may incorporate keyed geometry to maintain the rotational orientation of the mounted needle.

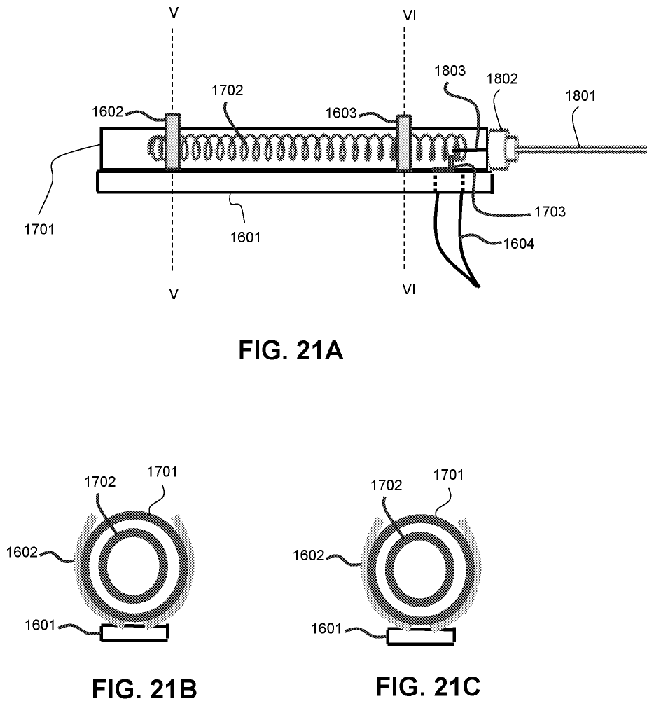

FIG. 21A depicts the gun 1600 carrying a removable subassembly including evacuation/extraction chamber 1701, tension spring 1702, and release mechanism 1703, together with needle 1801, needle hub 1802, and stylet 1803. The stylet 1803 is shown in a deployed (distal) state prior to retraction.

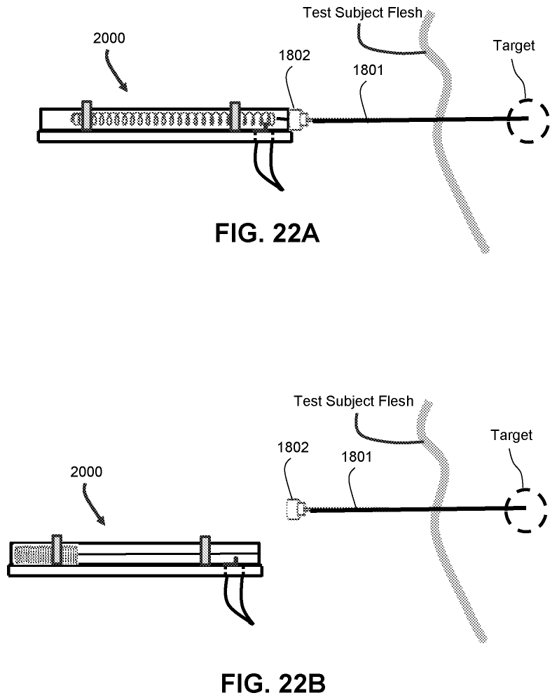

Sections V-V and VI-VI (FIGS. 21B-21C) show how clips 1602/1603 secure chamber 1701 and align release mechanism 1703 with trigger 1604, so that a single trigger stroke 1604 releases spring 1702 to retract stylet 1803 into chamber 1701. Depending on version, continued trigger travel may convey limited axial motion to chamber 1701 or associated elements; in capillary only variants, the chamber remains static after retraction. The mounting also facilitates quick detachment of the chamber while preserving needle placement (see FIGS. 22A-22B).

FIG. 22A shows a gun/extraction chamber assembly 2000 attached to needle 1801/needle hub 1802 with the needle inserted into a test subject at a target. In this condition the stylet is retracted and the operator may stabilize or manipulate the gun as needed.

FIG. 22B illustrates decoupling of the gun/extraction chamber assembly 2000 from the needle/hub 1801/1802 while the needle remains in situ at the target, enabling the operator to perform unobstructed capillary aspiration or fenestration with minimal disturbance. The needle can then be removed for specimen processing.

In the embodiments illustrated in FIGS. 20A-21C, the reusable trigger gun 1600 is shown with a proximal attachment clip 1602 and a distal attachment clip 1603 for receiving and securing an extraction chamber/needle assembly. However, the particular clip-based mounting arrangement is presented merely as one nonlimiting example. Any suitable mechanical, magnetic, friction-fit, keyed, or quick-release interface may be used, provided that it securely retains the extraction chamber during needle insertion and permits deliberate decoupling when desired. The geometry, material, and operative engagement of the attachment members are accordingly a matter of design choice well within the ordinary skill in the art.

Similarly, although the figures depict the trigger 1604 mechanically interfacing with a specific release mechanism 1703 associated with the extraction chamber 1701, the manner of operatively coupling the trigger to the extraction chamber release mechanism is not limited to the illustrated arrangement. A person having ordinary skill in the art will readily appreciate that a wide variety of couplings may be used—including, without limitation, direct mechanical linkages, pivoting levers, pushrod interfaces, cam followers, sliding actuation bars, torsion transfer elements, magnetic couplings, or electrical or electromechanical release devices—so long as actuation of the trigger results in the controlled release of the stored energy element (e.g., spring 1702) and the resulting retraction of the stylet into the extraction chamber.

In all such variants, the trigger-to-release coupling may include optional features such as lost-motion segments, timed dwell positions, overtravel limits, safety interlocks, and feedback elements (audible or tactile), depending on ergonomic preference, manufacturing considerations, or safety requirements. The selection and arrangement of these components fall within the design discretion of those of ordinary skill in the art and do not limit the scope of the inventive concepts.

In the embodiments of FIG. 19 and FIGS. 20A-22B, the needle and needle hub (e.g., 1500/1501, 1801/1802) are shown as releasably engageable with an extraction chamber (e.g., 1503, 1701) so that, after stylet retraction, the extraction chamber component is decoupled while the needle remains inserted in the test subject. The specific couplings illustrated—whether realized by direct attachment at the hub, a chamberside receiver, or interfaces carried by a reusable gun—are nonlimiting examples provided for clarity of explanation. Ideally, however, the trigger should retract the stylet and “softly” decouple from the needle to avoid jostling and displacement of the needle tip.

More generally, the manner of coupling and uncoupling the needle assembly to and from the extraction chamber is a matter of design choice well within the purview of those of ordinary skill in the art. Suitable alternatives include, without limitation, bayonet fittings, keyed snapfits, pushbutton detents, sliding collars, quarterturn locks, threaded adapters, magnetic couplers with mechanical backups, and friction or collet-style receivers, provided the interface securely retains the components during insertion/manipulation and permits deliberate decoupling when desired. Optional features such as tactile or audible feedback, indexing for rotational orientation, one way lockouts to prevent unintended release, and two-step “affirmative” release actions may be incorporated as needed to meet ergonomic, sterility, and safety objectives.

In embodiments employing a reusable trigger gun (e.g., 1600 with clips 1602/1603), the coupling/uncoupling between the extraction chamber/needle subassembly and the gun is likewise not limited to the pictured clip geometry. Equivalent quick-attach/quick-release mechanisms—such as lever/latch cradles, dovetail slides, camover clamps, magnetic docks with hard stops, or keyed rails—may be substituted, provided they maintain operative alignment between the gun's trigger and the chamber's release interface during actuation and allow rapid, intentional removal for in situ decoupling workflows. The operative coupling of the gun's trigger to the extraction chamber's release is accordingly a routine design selection, and may be achieved via direct mechanical linkage, cam or lever translation, pushrod or cable, torsional transfer, magnetic coupling, or electrical/electromechanical actuation, as previously discussed for the trigger-to-release interface.

In addition, the trigger shown in this embodiment is for illustrative purposes, and may be in the form of a trigger, button, switch, or other mechanical mechanism to release the spring and activate the decoupling mechanism. The location on the device is also for illustrative purposes, as it may be located anywhere along the reusable gun to suit ergonomic preferences. For example, ideal trigger location may vary with the length of the attached FNA needle.

Enumerated below are some notes directed to operation, advantages, and variations of the embodiments of FIGS. 19-22B.
1. Singlehanded retraction with continuous ultrasound control. The operator inserts the needle with the stylet deployed, actuates the trigger (1505 or 1604) to retract the stylet into chamber 1503 or 1701, and decouples the chamber while the other hand remains on the ultrasound probe—reducing target loss and overall procedure time.
2. In situ decoupling for capillary sampling. Both the integrated chamber of FIG. 19 and the gun mounted system of FIGS. 21-22B support detachment of the extraction chamber while the needle remains in the target, leaving only a slender needle in place for delicate capillary passes.
3. Reusable gun with disposable chamber/needle. The gun 1600 is reusable, whereas the chamber/needle subassemblies are designed for single use, facilitating rapid turnover with sterile disposables. Clips 1602/1603 enable fast load/unload and standardized alignment with the gun's trigger 1604 regardless of the internal spring type.
4. Energy storage options. Although tension springs (1504, 1702) are pictured, the retraction force can be generated by compression springs, torsion elements, or magnetically/electrically latched drivers, provided that the trigger-initiated release retracts the stylet fully into its chamber and permits in situ detachment.
5. Interchangeability and compatibility. The needle hubs 1501/1802 may use standard or keyed couplings; the chamber couplings can mechanically and sterically interlock with the hubs to ensure secure attachment during insertion and predictable decoupling after retraction. The support body 1601 can be adapted for varying needle gauges and lengths.
6. Safety lockout after retraction. In some embodiments, the release mechanism locks out once the stylet is retracted so that the stylet cannot be inadvertently redeployed. The lockout may be achieved by a unidirectional latch, pawl-and-ratchet, overcenter linkage, or equivalent geometry integrated into the release mechanism 1703 or the trigger 1604/1505 interface. In certain versions, a manual override requiring deliberate two-step input may be provided for servicing.
7. Affirmative decoupling to prevent unintended separation. The decoupling interface between the extraction chamber (1503, 1701) and the needle hub 1501/1802 and/or gun clips 1602/1603 may require affirmative operator input (e.g., squeeze tabs, sliding collar, or pushbutton detent) to prevent accidental separation during insertion or manipulation. The interface can include tactile and/or audible feedback to confirm coupling and decoupling events.
8. Capillary only vs. limited aspiration variants. In capillary-only embodiments, the extraction chamber 1701 remains substantially static after stylet retraction. In alternative embodiments, the chamber or an associated shuttle may be configured for a limited translation stroke relative to support body 1601 to generate limited negative pressure, providing a hybrid mode that augments capillary draw while maintaining a compact form factor. Stroke length and rate may be defined by internal stops or biasing elements to tailor aspiration magnitude for specific tissues.
9. Adjustable alignment and dwell control. The clips 1602/1603 may provide multiple indexed positions to adjust needle orientation, and the release mechanism may incorporate adjustable dwell or lost motion features to control the exact timing between trigger actuation and stylet retraction (e.g., to synchronize with ultrasound confirmation).
10. Materials and disposability. The gun 1600 can be formed from sterilizable engineering polymers or metals for repeated reuse, while the extraction chamber/needle subassemblies are fabricated as single-use sterile disposables with integrated tamper-evident features. Coupling geometries may be standardized to accommodate third-party needles via adapter hubs.

As an example, a sequence of use of the syringe-less embodiments herein may first include mounting a sterile extraction chamber/needle subassembly to clips 1602/1603 of gun 1600, and confirm release mechanism 1703 interfaces properly with trigger 1604. The needle 1801 with stylet 1803 is then deployed to the target under ultrasound guidance. Once target deployment is achieved, the trigger 1604 is actuated to release spring 1702 and retract stylet 1803 into chamber 1701. The gun/chamber is then decoupled from needle/hub 1801/1802 while the needle remains in situ, and then capillary aspiration is performed. A fresh chamber/needle gun-mounted unit is then re-attached for additional passes.

While embodiments of the inventive concepts have been described above with reference to drawings, it will be apparent to those of ordinary skill in the art that various changes and modifications may be made thereto without departing from the spirit and scope of the inventive concepts.

1-21. (canceled)

22. A syringeless fine needle aspiration device comprising:
a needle and a needle hub;
an extraction chamber;
a stylet movable between a deployed position extending through the needle and a retracted position received within the extraction chamber;
an energy-storing element coupled to the stylet; and
an operator actuated trigger integrated with the extraction chamber and operatively coupled to a release mechanism;
wherein actuation of the trigger releases the energy-storing element to retract the stylet into the extraction chamber; and
wherein the extraction chamber is releasably attachable to the needle hub such that the extraction chamber can be detached from the needle hub while the needle remains inserted in a test subject.

23. The device of claim 22, wherein the energy-storing element comprises a spring selected from a tension spring and a compression spring.

24. The device of claim 22, wherein the release mechanism comprises at least one of a latch, pawl-and-detent, overcenter linkage, cam-based detent, or an electrically or magnetically actuated catch.

25. The device of claim 22, further comprising a safety lockout configured to prevent redeployment of the stylet after retraction into the extraction chamber.

26. The device of claim 22, wherein at least the releasable attachment between the extraction chamber and the needle hub requires affirmative operator input to decouple, thereby inhibiting unintended separation during insertion or manipulation.

27. The device of claim 22, wherein the extraction chamber and the needle hub include keyed or indexed interfaces that maintain rotational orientation upon coupling.

28. The device of claim 22, further comprising a spring clamp within the extraction chamber that grips a proximal portion of the stylet during retraction.

29. The device of claim 22, wherein the releasable attachment provides at least one of tactile or audible feedback indicative of coupling and decoupling events.

30. The device of claim 22, wherein the extraction chamber or a shuttle associated therewith is configured for a limited translation stroke to generate limited negative pressure following stylet retraction.

31. A system comprising:
a reusable trigger gun having a support body and a trigger; and
a removable extraction chamber/needle subassembly comprising an extraction chamber, an energy-storing element, a release mechanism configured to interface with the trigger, a needle hub, a needle, and a stylet;
wherein the extraction chamber/needle subassembly is detachably mounted to the reusable trigger gun, and
wherein actuation of the trigger releases the energy-storing element to retract the stylet into the extraction chamber, and
wherein the extraction chamber/needle subassembly is detachable from at least the needle hub such that the subassembly can be detached while the needle remains inserted in a test subject.

32. The system of claim 31, wherein the detachable mounting comprises a keyed or friction-fit engagement, a bayonet interface, a latch, a sliding rail, or a magnetic/mechanical hybrid coupling that maintains operative alignment between the trigger and the release mechanism.

33. The system of claim 31, wherein the detachable mounting includes indexing features configured to set rotational orientation of the mounted subassembly.

34. The system of claim 31, wherein the reusable trigger gun further comprises a trigger safety configured to inhibit actuation until deliberately disengaged by an operator.

35. The system of claim 31, wherein the detachable mounting requires affirmative operator input to release the subassembly to prevent unintended separation during use.

36. The system of claim 31, wherein the reusable trigger gun is reusable and the extraction chamber/needle subassembly is a single-use disposable component.

37. The system of claim 31, wherein the reusable trigger gun is configured to accept detachable extraction chamber/needle assemblies of differing needle gauges or lengths.

38. A method of performing fine needle aspiration without a syringe body, comprising: mounting a removable extraction chamber/needle subassembly to a reusable trigger gun; inserting a needle of the subassembly into a test subject with a stylet in a deployed position; actuating a trigger of the trigger gun to release a stored energy element and retract the stylet into an extraction chamber of the subassembly; and detaching the extraction chamber/needle subassembly from at least a needle hub while leaving the needle inserted in the test subject to permit capillary sampling.

39. The method of claim 38, further comprising maintaining continuous ultrasound visualization of the needle tip with an opposite hand of the operator during actuation and detachment.

40. The method of claim 38, wherein releasing the stored energy element comprises actuating at least one of a latching release, a pawl-and-detent, an overcenter linkage, a cam-based detent, or an electrically or magnetically actuated catch.

41. The method of claim 38, wherein detaching the extraction chamber/needle subassembly requires affirmative operator input and produces at least one of tactile or audible feedback indicating successful decoupling while the needle remains in situ.

42-44. (canceled)
